(19) United States
(12) Patent Application Publication
(10) Pub. No.: US 2025/0186115 A1
(43) Pub. Date: Jun. 12, 2025

(54) COPD TARGETED LUNG DENERVATION (TLD)

(71) Applicant: Medtronic Cryocath LP, Toronto (CA)

(72) Inventors: Jean-Pierre Lalonde, Candiac (CA); Mark T. Stewart, Lino Lakes, MN (US); Joseph D. Brannan, Lyons, CO (US); Zhongping Yang, Woodbury, MN (US); Nicolas Coulombe, Anjou (CA); Robert C. Kowal, Excelsior, MN (US); Anthony W. Rorvick, Champlin, MN (US); Kurt R. Smith, Boulder (MN); William J. Dickhans, Longmont, CO (US); Randal C. Schulhauser, Phoenix, AZ (US)

(21) Appl. No.: 18/843,382

(22) PCT Filed: Mar. 30, 2023
(86) PCT No.: PCT/CA2023/050428

§ 371 Date: Sep. 3, 2024

Related U.S. Application Data
(60) Provisional application No. 63/326,981, filed on Apr. 4, 2022.

(51) Int. Cl. A61B 18/18; A61B 1/267; A61B 18/00; A61B 18/14

(52) U.S. Cl. CPC A61B 18/1815; A61B 1/2676; A61B 18/1492; A61B 2018/0016; A61B 2018/00166; A61B 2018/0022; A61B 2018/00434; A61B 2018/00541; A61B 2018/00577; A61B 2018/00773; A61B 2018/00982; A61B 2018/1467; A61B 2018/1861

(57) ABSTRACT

A medical system for targeted lung denervation includes an elongate shaft having a first lumen having a first length and sized and configured to be advanced through a trachea of a mammal, and a second lumen having a second length longer than the first length and sized and configured to be advanced into a target bronchus of the mammal, the second lumen further including a first expandable member at its distal end. A treatment device having a treatment element is included. The treatment device is sized and configured to be advanced through the second lumen and to denervate the target bronchus.

### CROSS-REFERENCE TO RELATED APPLICATION

The present application is related to and claims benefit under 35 U.S.C. § 119(e) from U.S. Provisional Patent Application Ser. No. 63/326,981, filed 4 Apr. 2022, entitled “COPD TARGETED LUNG DENERVATION (TLD)”, the entire contents of which being incorporated herein by reference.

### FIELD

The present technology is generally related to denervation of lung tissue for the treatment of chronic obstructive pulmonary disease (COPD).

### BACKGROUND

COPD is a chronic inflammatory lung disease that causes obstructed airflow from the lungs. Symptoms include breathing difficulty, cough, mucus production and wheezing. People with COPD are at increased risk of developing heart disease, lung cancer, and other diseases. Targeted lung denervation (TLD) is a surgical procedure in which a catheter is advanced through a bronchoscope to a target area within a target bronchus to denervate the target area. However, use of a bronchoscope to deliver the catheter limits the size of the catheter and constrains potential functionality of the catheter. For example, typical bronchoscopes are longer than necessary to reach the primary bronchus. As a result, a longer catheter may be needed, which reduces efficacy of therapeutic deliveries from the catheter.

### SUMMARY

The techniques of this disclosure generally relate to denervation of lung tissue for the treatment of chronic obstructive pulmonary disease.

In one aspect, the present disclosure provides a medical system for targeted lung denervation which includes an elongate shaft that has a first lumen having a first length and is sized and configured to be advanced through a trachea of a mammal, and a second lumen having a second length longer than the first length and is sized and configured to be advanced into a target bronchus of the mammal. The second lumen further includes a first expandable member at its distal end. A treatment device that has treatment element is included. The treatment device is sized and configured to be advanced through the second lumen and to denervate the target bronchus.

In another aspect of this embodiment, the treatment element includes a second expandable member configured to exchange ablative energy with the target bronchus.

In another aspect of this embodiment, the treatment element includes a second expandable member and a microwave radiator disposed within the second expandable member.

In another aspect of this embodiment, the treatment element is disposed between a pair of baluns disposed on the energy delivery device, and wherein the microwave radiator is configured to create toroidal shaped lesions within the target bronchus.

In another aspect of this embodiment, wherein, when inflated, the first expandable member is configured to provide anatomical centering of the second expandable member within the target bronchus.

In another aspect of this embodiment, the system further includes a bronchoscope sized and configured to be advanced through the elongate shaft.

In another aspect of this embodiment, the treatment element includes a plurality of electrodes configured to deliver radiofrequency energy to ablate the target bronchus.

In another aspect of this embodiment, a plurality of electrodes on the first expandable member or on the treatment device configured to determine if the target bronchus has been denervated.

In another aspect of this embodiment, the treatment device includes at least one sensor on its exterior surface, the sensor being configured to at least one from the group consisting of detect a presence of blood, measure tissue impedance, measure tissue temperature, measure tissue optical absorption or optical reflection.

In one aspect, a medical system for targeted lung denervation includes an elongate shaft having a first lumen having a first length and sized and configured to be advanced through a trachea of a mammal, and a second lumen having a second length longer than the first length and sized and configured to be advanced into a target bronchus of the mammal. The first lumen includes a cuff sized and configured to be inflated around both the first and second lumens and to create seal within the trachea when inflated. The second lumen further includes a first expandable member at its distal end. A treatment device that has a treatment element at its distal end is included. The treatment device is sized and configured to be advanced through the second lumen and to denervate the target bronchus.

In another aspect of this embodiment, the treatment element includes a second expandable member configured to exchange ablative energy with the target bronchus.

In another aspect of this embodiment, the treatment element includes a second expandable member and a microwave radiator disposed within the second expandable member.

In another aspect of this embodiment, the treatment element is disposed between a pair of baluns disposed on the treatment device, and wherein the microwave radiator is configured to create toroidal shaped lesions within the target bronchus.

In another aspect of this embodiment, wherein, when inflated, the first expandable member is configured to provide anatomical centering of the second expandable member within the target bronchus.

In another aspect of this embodiment, the system further includes a bronchoscope sized and configured to be advanced through the elongate shaft.

In another aspect of this embodiment, the treatment element includes a plurality of electrodes configured to deliver radiofrequency energy to ablate the target bronchus.

In another aspect of this embodiment, the treatment element further includes a plurality of electrodes on the first expandable member or on the treatment device configured to determine if the target bronchus has been denervated.

In another aspect of this embodiment, the system provides a means of applying stimulation to the bronchial nerves to allow for endpoint detection of nerve function. Stimulation at for example, 50 Hz applied to the nerves from electrodes in the bronchus may elicit a response which constricts the airways such that the pressure-air flow relationship is altered. Post-treatment, such an evaluation would provide an indication of procedural success. Such a system would provide means to monitor the airflow and air pressure during such testing

In one aspect, a medical system for targeted lung denervation includes an elongate shaft that has a first lumen having a first length and is sized and configured to be advanced through a trachea of a mammal, and a second lumen that has a second length longer than the first length and is sized and configured to be advanced into a target bronchus of the mammal. The first lumen includes a cuff at its distal end sized and configured to be inflated around both the first and second lumens and to create seal within the trachea when inflated. The second lumen further includes a first expandable member at its distal end. A treatment device having a treatment element at its distal end is included. The treatment device is sized and configured to be advanced through the second lumen and to denervate the target bronchus.

In one aspect, a method for targeted lung denervation includes advancing an elongate shaft through a trachea of a mammal and into a target bronchus, the elongate shaft includes a first lumen that has a first length and a second lumen that has a second length longer than the first length. The elongate shaft includes an inflatable cuff sized and configured to be inflated around both the first and second lumens and to create a seal within the trachea when inflated, and a first expandable member. The inflatable cuff is inflated to occlude the trachea. The first expandable member is inflated to contact a wall of the target bronchus. A treatment device that has a treatment element is advanced through the second lumen and into or through the first expandable member. The treatment element is configured to exchange thermal energy with the target bronchus. Thermal energy is exchanged with the target bronchus to denervate the target bronchus.

In another aspect of this embodiment, when inflated, the first expandable member is configured to provide anatomical centering of the treatment device within the target bronchus.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1A is a side view of a medical system constructed in accordance with the principles of the present application;

FIG. 1B is a side view of the medical system shown in FIG. 1A with a medical device being advanced through the medical system;

FIG. 1C is a side view of the medical system shown in FIG. 1B with the medical device being advanced distally of the medical system and a treatment element inflated;

FIG. 2 is a side view of another medical device constructed in accordance with the principles of the present application;

FIG. 3 is a side view of another medical device constructed in accordance with the principles of the present application;

FIG. 4 is a side view of another medical device constructed in accordance with the principles of the present application;

FIG. 5 is a side view of another medical device constructed in accordance with the principles of the present application; and

FIG. 6 is a flow chart showing the steps of an exemplary targeted lung denervation.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

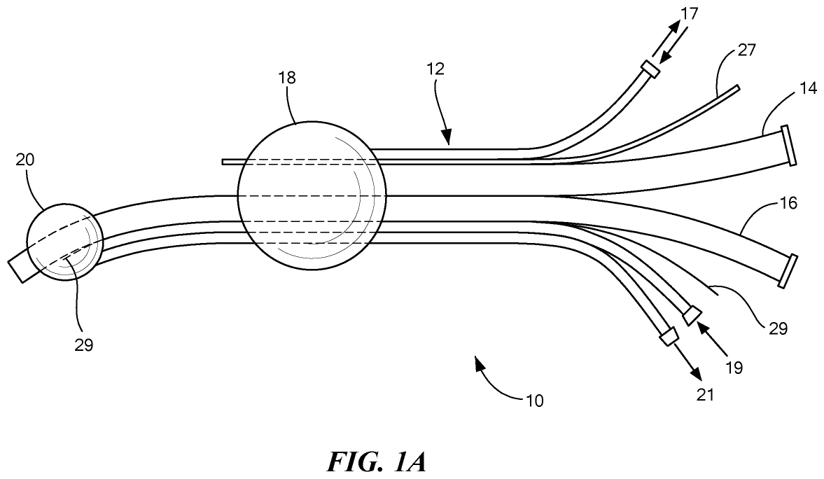

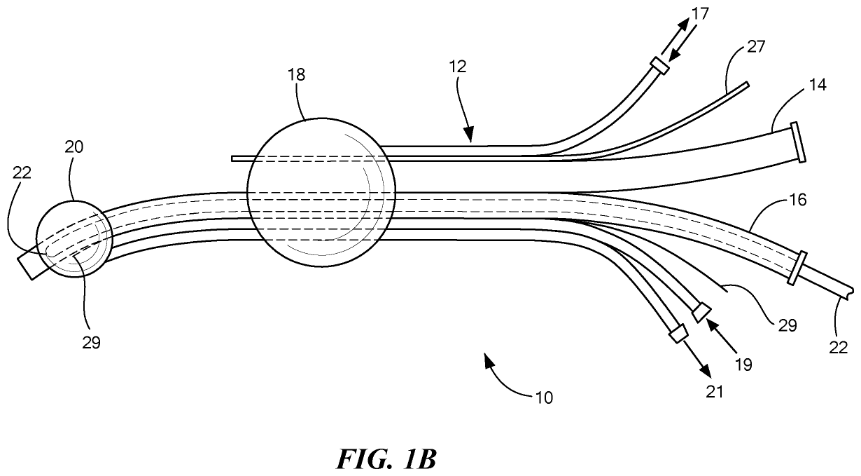

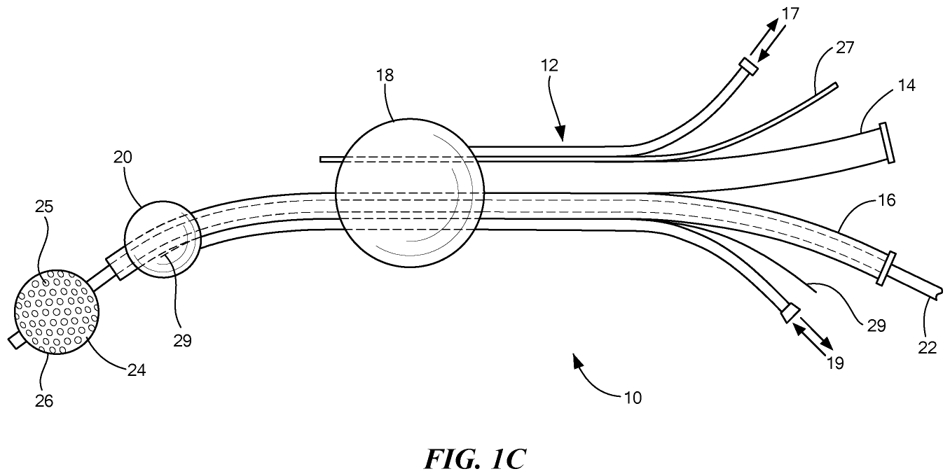

Referring now to the drawings in which like reference designators refer to like elements, there is shown in FIG. 1A an exemplary medical system for targeted lung denervation (TLD) constructed in accordance with the principles of the present application and designated generally as “10.” The medical system 10 is configured to be utilized inside the trachea and lungs of a mammalian patient for TLD. In one configuration, the medical system 10 includes an elongate shaft 12 having a first lumen 14. The first lumen 14 has a first length and is sized and configured to be advanced through a trachea of a mammal. In particular, the first length is such that the first lumen 14 terminates within the trachea. In the configuration shown in FIG. 1A, the elongate shaft 12 may be flexible and include a splitable sheath. A second lumen 16 having a second length longer than the first length is sized and configured to be advanced into a target bronchus of the mammal. For example, the second lumen 16 may be disposed in a side-by-side relationship with the first lumen 14 within the elongate shaft 12 and may terminate in a target bronchus. In one configuration, the second lumen 16 or the first lumen 14 includes an inflatable cuff 18 at the distal end of the first lumen 14. The cuff 18 is configured to surround both the first lumen 14 and the second lumen 16 when inflated to create a seal within the trachea. In the configuration, shown in FIGS. 1A-1C, the elongate shaft 12 may include a lumen 17 for inflation and/or deflation of the inflatable cuff 18. As shown in FIG. 1C, in some configurations, the lumen 17 may be configured for bidirectional flow, thus allowing for inflation and deflation of the cuff 18 through the use of a single lumen 17. The lumen 17 maybe be coupled to a fluid source, for example, saline or air. In one configuration, the second lumen 16 further includes a first expandable member 20 at its distal end in other configurations the first expandable member 20 is not included. The first expandable member 20 may be a balloon that is sized and configured to contact the wall of the target bronchus when inflated to create a seal within the target bronchus. In the configuration shown in FIGS. 1A-1, the first expandable member 20 may be in fluid communication with an inlet 19 and outlet 21 for inflation and deflation respectively of the first expandable member 20.

A treatment device 22 having a treatment element 24 may be sized and configured to be advanced through the second lumen 16 and is configured to denervate the target bronchus. For example, the treatment element 24 may be configured to denervate the target bronchus with one or more of the following modalities: cryogenic denervation, radiofrequency denervation, or microwave denervation, as discussed in more detail below. For example, as shown in FIG. 1B, the treatment device 22 is a microwave catheter advanced through lumen 16 and configured to deliver microwave energy from within the first expandable member 20. Although a microwave catheter is shown as the treatment device 22 in FIG. 1B, it is contemplated that refrigerant may be circulated within the first expandable member 20, instead of advancing the microwave catheter, to denervate the bronchus by extracting heat from the target tissue via cryogenic energy. For example, the inlet and outlet indicated in FIG. 1B to be in fluid communication with the first expandable member 20 may be in fluid communication with a cryogenic fluid source (not shown) to inflate the first expandable member 20 to denervate the target bronchus.

In FIG. 1C, the treatment device 22 includes a second expandable member 26 at its distal end as the treatment element 24. In one configuration, the second expandable member 26 is sized to be received within the first expandable member 20. In some configurations, the treatment device 22 includes an inflation lumen 23 and an exhaust lumen 25 in fluid communication with the cryogenic fluid source (not shown). The cryogenic fluid is circulated through the treatment device 22 toward the second expandable member 26 to denervate the target bronchus via the inflation lumen 23, and is exhausted from the expandable member 26 via the exhaust lumen 25. In other configurations, the second expandable member 26 extends distally to the first expandable member 20 and is configured to ablate the target bronchus with, for example, cryogenic, radiofrequency (RF), or microwave energy. In some such configurations, the first expandable member 20, when inflated, is used to provide mechanical support/anatomical centering within a target bronchus for placement of the treatment element 24 and/or second expandable member 26. Additionally, in some configurations, the second expandable member 26 or the first expandable member includes a plurality of electrodes 25 circumscribing the second expandable member 26 to ablate the target bronchus with radiofrequency (RF) energy. In another configuration, the plurality of electrodes 25 may further be configured to confirm expandable member 26 contact with tissue, determine ablation progression and success for example, via bronchial nerve stimulation, impedance monitoring, and/or surface temperature measurements. A fiber optic camera or bronchoscope 27 may be included within the elongate shaft 12 and is sized to be received within either the first lumen 14 or the second lumen 16 or adjacent to the first lumen 14 or the second lumen 16 to visualize the placement of the first expandable member 20. Optionally, a thermocouple 29 or other sensor may be included and sized to be received adjacent to the first expandable member 20 to measure a temperature of the first expandable member 20, to confirm tissue contact or to support treatment progress monitoring.

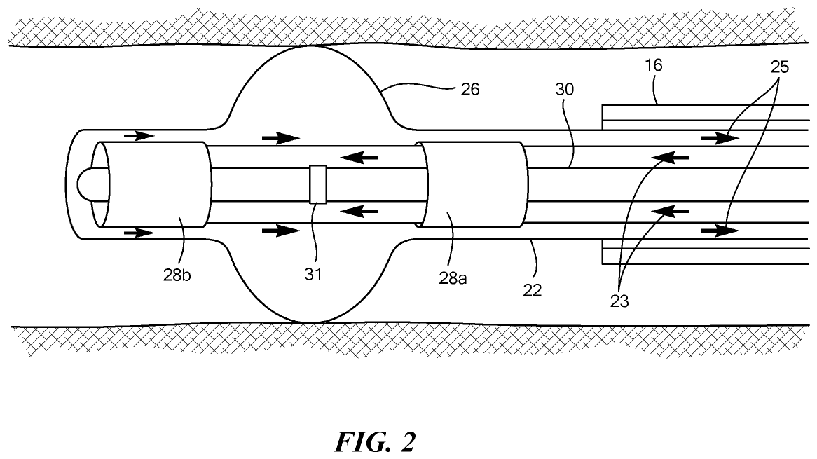

Referring now to FIG. 2, in another configuration, the treatment device 22 is a microwave catheter configured to deliver microwave energy to denervate the target bronchus. For example, the treatment device 22 may include the second expandable member 26 or first expandable member 20 disposed between a pair of baluns 28a and 28b which operate to focus on the radiated microwave energy to create substantially toroidal shape lesions to denervate the target bronchus. As shown in FIG. 2, a microwave radiator 31 is disposed on an elongate body 30 of the treatment device within the second expandable member 26. The elongate body 30 may be fixed or movable with respect to the second expandable member 26 to create a desired lesion.

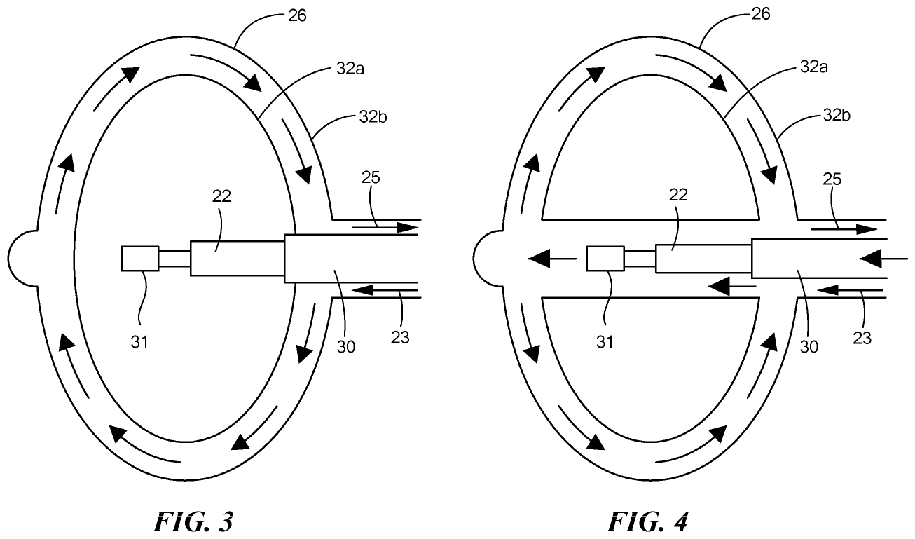

Referring now to FIG. 3, in another configuration, the treatment device 22 is a microwave catheter configured to deliver microwave energy to denervate the target bronchus. In the configuration shown in FIG. 3, the treatment device 22 includes the second expandable member 26, which includes a first balloon 32a disposed within a second balloon 32b. The first balloon 32a may be inflated by air and thus creates a pocket within which the microwave radiator 31 is disposed and emits microwave energy. Between the exterior surface of balloon 32a and the interior surface of balloon 32b is an irrigation lumen including a bolus of circulating saline configured to extract heat from the tissue as microwave energy is being applied and thus protecting non-targeted tissues in direct contact with the second expandable member 26 from thermal damage. In another configuration, as shown in FIG. 4, the microwave radiator 31 is disposed within the elongate body 30 of the treatment device 22 isolated from the first balloon 32a and the second balloon 32b which surrounds the elongate body 30.

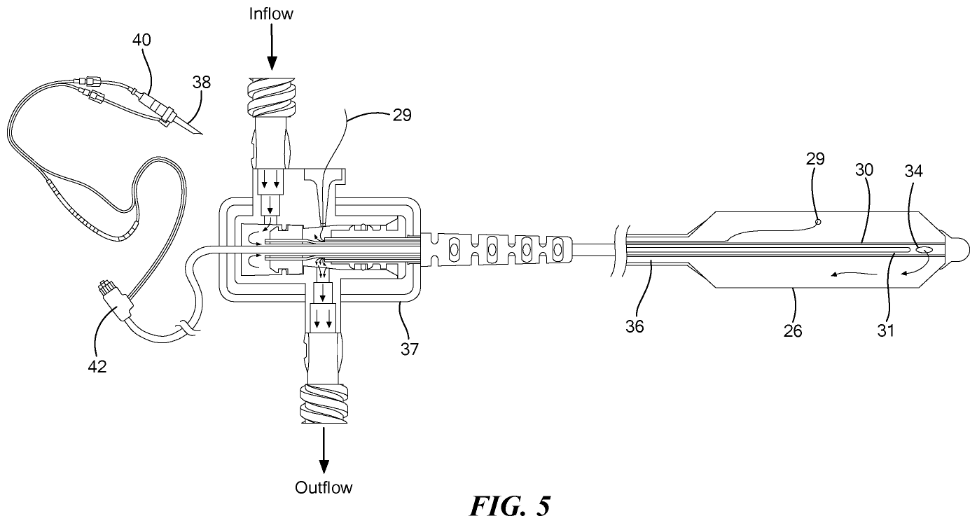

In yet another configuration, as shown in FIG. 5, the microwave radiator 31 is disposed within the elongate body 30, isolated the second expandable member 26, and irrigant circulates around the elongate body 30. In the configuration shown in FIG. 5, a single expandable member 26 is used, although multiple expandable members are contemplated. The thermocouple 29 may be disposed on the exterior of the second expandable member 26 to monitor a temperature of the second expandable member 26 to confirm a performance of the ablation. In this configuration, saline is used to irrigate the microwave radiator 31 and inflate the expandable member 26 with concentric inflow 34 and outflow lumens 36. To create sufficient backflow pressure to fill the second expandable member 26, the flow direction may be reversed. A flow restriction valve (not shown), may be included in communication with the outflow lumen 36 to vary pressure within the second expandable member 26. A hub 37 provides for an inflow connector labeled as “inflow” and an outflow connector labeled as “outflow,” which may connect to a vacuum source (not shown). In some configurations, the vacuum source may be a peristaltic pump configured to irrigate or otherwise exhaust fluid from the second expandable member 26. In one configuration, the outflow and inflow connectors are parallel to each other, but may be disposed at other positions in communication with the hub 37. The microwave catheter may further include an intravenous spike 38, a drip chamber 40 and a cable connector for engaging a microwave generator (not shown).

As described herein, it is to be understood that the configurations of the medical device shown in FIGS. 2-5, constructed with the principles of the present application, may be used independently of the configurations shown in FIGS. 1A, 1B, and 1C, and are not limited to use in a medical system for targeted lung denervation (TLD). In other words, the device of FIGS. 2-5 may be used in a variety of additional medical procedures not described in detail herein.

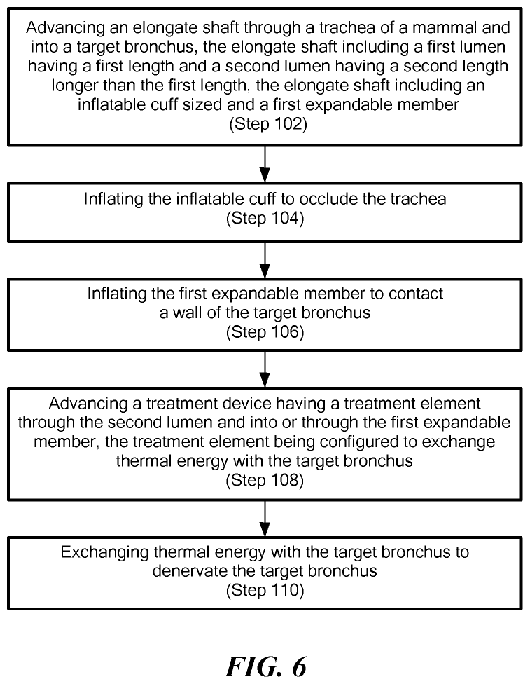

Referring now to FIG. 6, in an exemplary method of performing a TLD, the elongate shaft 12 of the medical system 10 is advanced though the mammal's trachea and into a target bronchus (Step 102). The inflatable cuff 18 is inflated within the trachea to provide a seal therein and occlude the trachea (Step 104). For example, the inflatable cuff 18 may be in fluid communication with a fluid source, for example, air or saline, to inflate the inflatable cuff 18. The first expandable member 20 is inflated within the target bronchus to contact the wall of the target bronchus (Step 106). For example, the first expandable member 20 may be in fluid communication with a fluid source, for example, air or saline, to inflate the first expandable member 20. The treatment element 22 of the treatment device 24 is advanced through the elongate shaft 12 and into or through the first expandable member 20 (Step 108). For example, as discussed above, the medical system 10 may include a second lumen 16 through which the treatment element 22 is advanced. Thermal energy is exchanged between the treatment element 22 and the target bronchus to denervate the target bronchus (Step 110). For example, as discussed above, such thermal energy may include microwave, radiofrequency, or cryogenic energy.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A medical system for targeted lung denervation, comprising:
an elongate shaft having a first lumen having a first length and sized and configured to be advanced through a trachea of a mammal, and a second lumen having a second length longer than the first length and sized and configured to be advanced into a target bronchus of the mammal, the second lumen further including a first expandable member at its distal end; and
a treatment device having a treatment element, the treatment device being sized and configured to be advanced through the second lumen and to denervate the target bronchus.

2. The system of claim 1, wherein the treatment element includes a second expandable member configured to exchange ablative energy with the target bronchus.

3. The system of claim 1, wherein the treatment element includes a second expandable member and a microwave radiator disposed within the second expandable member.

4. The system of claim 3, wherein the treatment element is disposed between a pair of baluns disposed on the treatment device, and wherein the microwave radiator is configured to create disc shaped lesions within the target bronchus.

5. The system of claim 3, wherein, when inflated, the first expandable member is configured to provide anatomical centering of the second expandable member within the target bronchus.

6. The system of claim 1, further including a bronchoscope sized and configured to be advanced through the elongate shaft.

7. The system of claim 1, wherein the treatment element includes a plurality of electrodes configured to deliver radiofrequency energy to the target bronchus.

8. The system of claim 1, further including a plurality of electrodes on the treatment device configured to determine if the target bronchus has been denervated.

9. The system of claim 1, wherein the treatment device includes at least one sensor on its exterior surface to detect a presence of blood.

10. A medical system for targeted lung denervation, comprising:
an elongate shaft having a first lumen having a first length and sized and configured to be advanced through a trachea of a mammal, and a second lumen having a second length longer than the first length and sized and configured to be advanced into a target bronchus of the mammal;
the first lumen including a cuff sized and configured to be inflated around both the first and second lumens and to create seal within the trachea when inflated;
the second lumen further including a first expandable member at its distal end; and
a treatment device having a treatment element at its distal end, the treatment device being sized and configured to be advanced through the second lumen and to denervate the target bronchus.

11. The system of claim 10, wherein the treatment element includes a second expandable member configured to exchange ablative energy with the target bronchus.

12. The system of claim 10, wherein the treatment element includes a second expandable member and a microwave radiator disposed within the second expandable member.

13. The system of claim 12, wherein the treatment element is disposed between a pair of baluns disposed on the treatment device, and wherein the microwave radiator is configured to create toroidal shaped lesions within the target bronchus.

14. The system of claim 13, wherein, when inflated, the first expandable member is configured to provide anatomical centering of the second expandable member within the target bronchus.

15. The system of claim 10, further including a bronchoscope sized and configured to be advanced through the elongate shaft.

16. The system of claim 10, wherein the treatment element includes a plurality of electrodes configured to deliver radiofrequency energy to the target bronchus.

17. The system of claim 10, further including a plurality of electrodes on the treatment device configured to determine if the target bronchus has been denervated.

18. A medical system for targeted lung denervation, comprising:
an elongate shaft having a first lumen having a first length and sized and configured to be advanced through a trachea of a mammal, and a second lumen having a second length longer than the first length and sized and configured to be advanced into a target bronchus of the mammal;
the first lumen including a cuff at its distal end sized and configured to be inflated around both the first and second lumens and to create seal within the trachea when inflated;
the second lumen further including a first expandable member at its distal end; and
a treatment device having a treatment element at its distal end, the treatment device being sized and configured to be advanced through the second lumen and to denervate the target bronchus.

19. A method for targeted lung denervation, comprising:
advancing an elongate shaft through a trachea of a mammal and into a target bronchus, the elongate shaft including a first lumen having a first length and a second lumen having a second length longer than the first length, the elongate shaft including an inflatable cuff sized and configured to be inflated around both the first and second lumens and to create seal within the trachea when inflated, and a first expandable member;
inflating the inflatable cuff to occlude the trachea;
inflating the first expandable member to contact a wall of the target bronchus;
advancing a treatment device having a treatment element through the second lumen and into or through the first expandable member, the treatment element being configured to exchange thermal energy with the target bronchus; and
exchanging thermal energy with the target bronchus to denervate the target bronchus.

20. The method of claim 19, wherein, when inflated, the first expandable member is configured to provide anatomical centering of the treatment device within the target bronchus.
